(19) United States
(12) Patent Application Publication
(10) Pub. No.: US 2020/0345588 A1
(43) Pub. Date: Nov. 5, 2020

(54) MONITOR DEVICE FOR REAL TIME COMPLIANCE INSTRUCTIONS AND USER FEEDBACK AND CLINICIAN COMMUNICATION

(71) Applicant: THE TRUSTEES OF INDIANA UNIVERSITY, Indianapolis, IN (US)

(72) Inventors: Jonathan G. MERRELL, Bloomington, IN (US); Meagan B. O'NEILL, Zionsville, IN (US); Ezueli C. DOS SANTOS, Zionsville, IN (US); Omar N. NEZAMUDDIN, Indianapolis, IN (US)

(21) Appl. No.: 16/962,016

(22) PCT Filed: Jan. 15, 2019
(86) PCT No.: PCT/US2019/013620

§ 371 Date: Jul. 14, 2020

Related U.S. Application Data
(60) Provisional application No. 62/617,823, filed on Jan. 16, 2018.
(60) Provisional application No. 62/749,332, filed on Oct. 23, 2018.

(51) Int. Cl. A61J 7/04; A61M 15/00; G16H 20/10; G16H 40/67

(52) U.S. Cl. CPC A61J 7/0427; A61J 7/0418; A61J 7/049; A61M 15/0086; A61J 2200/30; G16H 20/10; G16H 40/67; A61J 2205/60; A61J 2200/70; A61M 15/009

(57) ABSTRACT

A self-administered medicament delivery device system that desirably alert a clinician to improper and/or suboptimal patient use or compliance, and instructs and affirms the user on proper use in realtime. The invention improves the state of the art by proactively teaching and aiding users on proper techniques so the user obtains the most benefit from their device. By providing training, feedback, and reporting to both the user and the clinician, the invention allows users to make corrections immediately and allows clinicians to more accurately assess if medicine or technique is the problem in a patient whose condition is not remediated by a treatment regimen.

### STATEMENT OF GOVERNMENTAL RIGHTS

This invention was made with government support under TR001108 awarded by the National Institutes of Health. The government has certain rights in the invention.

Asthma and chronic obstructive pulmonary disease are significant causes of emergency room visits, hospitalizations, and death. This is often due to lack of patient understanding and adherence to their physician-prescribed treatment regimen. Many treatment regimens provide medicaments therapeutically in real time using rescue inhalers, as well as prophylactically using a controller inhaler. Both rescue and controller medications are highly effective, but in at least 50% of cases, patients or their parents do not completely comply with physician prescribed regimens. In reality 80-90% of patients do not employ proper techniques with their medicament dispensing device. Such noncompliance contributes to an estimated $100 billion annual burden in the United States alone.

“Smart inhalers” such as PROPELLER™, CARETRX™, COHERO™, CONNECTINH™, and ADHERIUM™ focus on recording the time that a dose of medication was taken and reminding the user to take doses; while helpful and perhaps augmented by a phone application that allows users to track their inhaler usage, one skilled in the art recognizes that actuating doses is only one aspect of compliant, efficacious delivery of medicament. A patient just taking doses regularly may not be getting the required benefit if their technique in using the device is wrong or is sub-optimal.

The invention improves the state of the art by proactively teaching and aiding users on proper techniques so the user obtains the most benefit from their device. By providing training, feedback, and reporting to both the user and the clinician, the invention allows users to make corrections immediately and allows clinicians to more accurately assess if medicine or technique is the problem in a patient whose condition is not remediated by a treatment regimen.

Embodiments of the invention include a medication delivery system that combines user input and sensor data to provide the user with real time auditory training, resulting in facilitating the user's mastery of a technique to administer a medication. The medication delivery system is applicable to various routes of administration including, but not limited to, medications taken by oral, intranasal, inhalation, transdermal, subcutaneous injection, intramuscular injection, intravenous infusion, enema, bladder catheter, pump, or intrathecal routes and combinations thereof. The inventive system is applicable to a pill-taking compliance device that dispenses in the home each day as medications are prescribed to be taken.

Another embodiment is a medication delivery system that includes a medication delivery device, and a monitor attached to the medication delivery device, where the monitor includes a speaker configured to provide, in real-time, at least one of medication use prompts, reminders, and feedback to at least the user and optionally also to a clinician caring for the user. The medication delivery system may include at least a first processor that is programmed in real time by the clinician with patient-specific instructions or altered patient specific instructions. The instructions may be related to medication type, dose quantity, dose volume, dose time, dose frequency, and combinations thereof. The system may further include a second processor that compares new instructions with previous instructions and transmits the comparison to a mobile device application.

A further embodiment is a medication delivery system where the medication delivery device is an inhaler that includes a canister housing and a user inhaler device, a spacer coupled to the user inhaler device, where the spacer includes at least one of a magnet, a radiofrequency identification (RFID) chip, or a nearfield communication (NFC) device; and where the monitor is removeably attached to the canister housing of the medication delivery device. The monitor may also include a sensor configured to sense the presence of the magnet, RFID chip or NFC device of the spacer and to indicate the presence of the sensor to a microcontroller mounted to the monitor. The inhaler may be a metered dose inhaler or a non-metered dose inhaler. The monitor attached to the medication delivery device may also include a motion sensor configured to detect at least one motion characteristic of use of the medication delivery device.

One more embodiment is a medication delivery system that includes a medication delivery device with a monitor attached to the medication delivery device, where the monitor includes a motion sensor configured to detect use of the medication delivery device and to respond to a signal indicating use of the medication delivery device by transitioning from a sleep mode to an operational mode.

Yet another embodiment includes a medication delivery system that includes a medication delivery device with a monitor attached to the medication delivery device, where the monitor includes a microphone configured to detect use of the medication delivery device.

Embodiments of the invention also include a medication monitoring system that includes:

a medication inhaler; a spacer coupled to the medication inhaler, the spacer comprising at least one magnet, radiofrequency identification (RFID) chip, a nearfield communication (NFC) device, or means to engage the spacer; a plurality of sensors configured to detect the spacer when the inhaler is in use; at least one accelerometer; at least one microphone; at least one sensor; at least one memory unit; at least one speaker; at least one transmitter; and at least one processor configured to receive data from the plurality of sensors.

Exemplary sensors include, but are not limited to, a capacitance sensor, a thermometer, an infrared light detector, an ambient light sensor, a pressure sensor, a humidity sensor, an impedance sensor, and combinations thereof. At least one of the plurality of sensors may sense when the medication is delivered from the inhaler. At least one of the plurality of sensors may be an accelerometer that detects qualitative and quantitative inhaler shaking before medication delivery from the inhaler. At least one of the plurality of sensors may detect the corresponding magnet or RFID chip or NFC device located in the spacer. The at least one processor may be programmed in real time with patient specific instructions or altered patient specific instructions. Exemplary instructions include, but are not limited to type of medication, dose quantity, dose volume, inhaler shaking, spacer presence, time of dose, and combinations thereof, and the processor generates a comparison of the instructions with data from the plurality of sensors and transmits the comparison to a mobile device application. The transmitter may be configured to receive data from the first processor and transmit the received data to a phone application and to the patient's caregiver or clinician.

Another embodiment is a method to determine correct use of an inhaler by a user that includes the steps of: providing a user with an inhaler comprising a medication monitoring device, the device comprising a plurality of sensors configured to transmit data to at least one processor, programming the at least one processor with user-specific instructions; determining whether the data concurs with the user-specific instructions, storing the data and alerting the user and/or clinician with nonconcurrance; and transmitting the data comparison, using a transmitter, to the user and clinician, where the data comparison indicates instruction compliance or non-compliance and provides suggestions for achieving compliance or affirmations of compliance. The user-specific instruction are from at least one of a clinician or a manufacturer. The clinician instructions are stored in at least one memory. The method may further include a first processor retrieving the stored data about medication dose administration time from the at least one memory. The processor transmits the data to the at least one transmitter to transmit a reminder to the user's mobile device. The plurality of sensors transmit recorded data to the at least one processor, and the processor stores the transmitted data in at least one memory. The at least one transmitter transmits the recorded data to both the user's mobile device and to an on-line data repository for clinician and/or researcher access. During data gathering and recordation by the sensors, the at least one processor compares the recorded data with the programmed instructions and determines if the recorded data aligns with the programmed instructions. Non-aligned data results in the at least one processor transmitting feedback to the user through the at least one speaker.

A further embodiment is a monitor unit configured to be coupled to a medication delivery device. The monitor unit includes a monitor unit body and a monitor unit central opening; a material capable of stretching to widen the monitor unit central opening to facilitate attachment of the monitor unit to the medication delivery device through the monitor unit central opening; at least one light source that performs a function selected from the group consisting of power indication or visual feedback to a user; a USB port to receive a USB cable; a port to receive a memory card; a sensor to detect if a mouth of a user has formed a seal around a mouthpiece; a reflector to facilitate communication between an infrared transmitter and an infrared receiver; at least one of a magnet, an infrared transmitter-emitter, a radiofrequency identification (RFID) chip or a mechanical button to detect a presence of a spacer; a speaker to provide auditory feedback to a user; and, at least one microphone to determine if a user is inhaling with proper force and/or to detect dose release. In one embodiment, the monitor unit further comprises at least one contact for supplying battery power to a spacer.

One more embodiment is a medication delivery device that includes at least one light source that performs a function selected from the group consisting of power indication or visual feedback to a user; a USB port to receive a USB cable; a port to receive a memory card; a sensor to detect if a mouth of a user has formed a seal around a mouthpiece; a reflector to facilitate communication between an infrared transmitter and an infrared receiver; at least one of a magnet, an infrared transmitter-emitter, a radiofrequency identification (RFID) chip a mechanical button or a capacitive sensor to detect a presence of a spacer; a speaker to provide auditory feedback to a user; and, at least one microphone to determine if a user is inhaling with proper force and/or to detect dose release. In one embodiment, the medication delivery device further comprising a temperature sensor.

Yet another embodiment is a spacer configured to be coupled to a medication inhaler where the spacer component includes: at least one magnet, radiofrequency identification (RFID) chip, a nearfield communication (NFC) device or a capacitive sensor. In one embodiment, the spacer includes an infrared transmitter-emitter. In one embodiment, the spacer includes a battery. In one embodiment, the spacer includes an accelerometer. In one embodiment, the spacer includes a capacitive sensor on a mouthpiece. In one embodiment, the spacer includes a reflector. In one embodiment, the spacer includes a microcontroller. In another embodiment, the spacer includes a memory/data storage. In a further embodiment, the spacer includes Bluetooth.

Embodiments of the invention also include a method to determine correct use of an inhaler by a user that includes the steps of: providing a user with an inhaler, the inhaler comprising a plurality of sensors configured to transmit data to at least one processor; programming the at least one processor with user-specific instructions; determining whether the data concurs with the user-specific instructions; storing the data and alerting the user and/or clinician with nonconcurrance; and transmitting the data comparison, using a transmitter, to the user and clinician, where the data comparison indicates instruction compliance or non-compliance and provides suggestions for achieving compliance or affirmations of compliance.

Another embodiment is a method to determine proper dose received and proper technique by a user is disclosed that includes the steps of: providing a user with a spacer to use with the inhaler, the spacer comprising a plurality of sensors configured to transmit data to at least one processor, programming the at least one processor with user-specific instructions; determining whether the data concurs with the user-specific instructions, storing the data and alerting the user and/or clinician with nonconcurrance; and transmitting the data comparison, using a transmitter, to the user and clinician, where the data comparison indicates instruction compliance or non-compliance and provides suggestions for achieving compliance or affirmations of compliance.

### BRIEF DESCRIPTION OF THE DRAWINGS

FIG. 1 is a perspective view of an embodiment of an inventive system.

FIG. 2 is a perspective view of an embodiment of an inventive monitor unit.

FIG. 3 is an orthogonal top view of an embodiment of an inventive monitor unit.

FIG. 4 is an orthogonal back view of an embodiment of an inventive monitor unit.

FIG. 5A, FIG. 5B and FIG. 5C are perspective views of an embodiment of an inventive monitor unit showing sequential steps of connecting the unit to a delivery device, FIG. 5A pre-connection, FIG. 5B partial connection, FIG. 5C connection.

FIG. 6A is a perspective view of an embodiment of an inventive system; FIG. 6B is a sample set up of the electronic components that can reside within the monitor unit; and, 6C is a perspective view of an embodiment of an inventive system.

FIG. 7A is an orthogonal front view of an embodiment of an inventive monitor unit. FIG. 7B is an orthogonal top view of an embodiment of an inventive monitor unit. FIG. 7C is an orthogonal bottom view of an embodiment of an inventive monitor unit.

FIG. 8 is an orthogonal side view of an embodiment of an inventive system.

FIG. 9A is a sample calendar view of an embodiment of an application showing compliance during a particular month. FIG. 9B is a sample weekly assessment of an embodiment of an application showing efficacy of patient compliance with a prescribed therapy regimen.

FIG. 10A shows an embodiment of a software algorithm for the patient APP; specifically, available programs and APP interaction with the device to provide patient feedback and assistance. FIG. 10B shows an embodiment of a software algorithm for the clinician application; specifically, available programs to monitor and facilitate patient compliance.

FIG. 11A shows a perspective view of an embodiment of an inventive system, and FIG. 11B shows a perspective view of an embodiment of an inventive system attached to a dry particle inhaler.

FIG. 12 shows a perspective view of an embodiment of an inventive system attached to a nebulizer.

FIG. 13 shows a perspective view of an embodiment of an inventive monitor unit.

FIG. 14 shows a perspective view of an embodiment of an inventive monitor unit.

FIG. 15 shows a perspective view of an embodiment of an inventive monitor unit.

FIG. 16 shows a perspective view of an embodiment of an inventive monitor unit.

FIG. 17 shows an orthogonal right side view of an embodiment of an inventive monitor unit.

FIG. 18 shows an orthogonal top side view of an embodiment of an inventive monitor unit.

FIG. 19 shows an orthogonal front side view of an embodiment of an inventive monitor unit.

FIG. 20 shows a perspective view of an embodiment of the invention.

FIG. 21 shows attached perspective view of an embodiment of an inventive monitor unit.

FIG. 22 shows an orthogonal rear side view of an embodiment of an inventive delivery device.

FIG. 23 shows a perspective view of an embodiment of an inventive delivery device.

FIG. 24 shows a perspective view of an embodiment of an inventive delivery device.

FIG. 25 shows an orthogonal right side view of an embodiment of an inventive delivery device.

FIG. 26 shows a perspective view of an embodiment of an inventive delivery device.

FIG. 27 shows a perspective view of an embodiment of an inventive spacer.

FIG. 28 shows a perspective view of an embodiment of an inventive spacer.

FIG. 29 shows a perspective view of an embodiment of an inventive spacer.

FIG. 30 shows a perspective view of an embodiment of an inventive spacer.

FIG. 31 shows a perspective view of an embodiment of an inventive spacer.

FIG. 32 shows a perspective view of an embodiment of an inventive spacer.

FIG. 33 shows a perspective view of an embodiment of an inventive spacer.

FIG. 34 shows an orthogonal rear side view of an inventive spacer.

FIG. 35 shows perspective view of an embodiment of an inventive spacer.

FIG. 36 shows an exploded view of an embodiment of an inventive delivery device.

FIG. 37 shows an orthogonal right side view of an embodiment of an inventive delivery device.

One skilled in the art appreciates that the inventive embodiments can be implemented in hardware, software, and/or firmware. Programming code according to the embodiments can be implemented in any viable programming language such as C, C++, Python, HTML, XTML, JAVA or any other viable high-level programming language, or a combination of a high-level programming language and a lower level programming language.

Programming can be accomplished in real time by a physician, either in-person by a wired connection or remotely via a wireless connection, and can be altered in real time to meet patient needs and/or clinician changes. For example, the microcontroller can be programmed to give different instructions to patients, depending on what medication is prescribed. For example, if a clinician changes a patient's medication form, e.g., FLOVENT™ to QVAR™, then the microcontroller is programmed to provide the user with instructions specific to QVAR™. The device can incorporate manufacturer instructions that have been tailored by the clinician to a user's specific regimen.

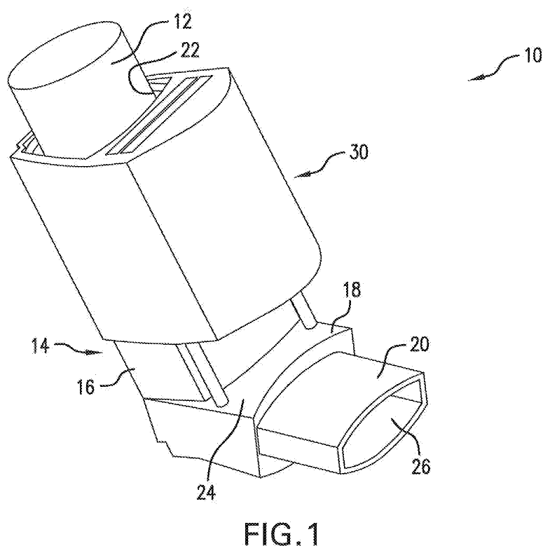

FIG. 1 shows a metered dose inhaler 10 that generally includes a pressurized canister 12 and a delivery device 14. In many inhalers, canister 12 is an aerosol canister made of aluminum or stainless steel including medication along with a liquefied gas propellant, and in some cases, stabilizing excipients. The lower end of canister 12 (not shown) generally includes a metering valve that is actuated by a mechanism (not shown) within delivery device 14 when canister 12 is actuated, e.g., by compression or by another actuation mechanism. As a result, a medicament aerosol is released from canister 12 in a controlled manner to deliver a specific quantity of the medicament to the user. The user inhales the medicament aerosol for delivery to the lungs.

Delivery device 14 generally includes a canister housing 16, a delivery chamber 18, and a mouthpiece 20. Canister housing 16 includes a central opening 22 sized to receive canister 12. Delivery chamber 18 is in flow communication with the output of the metering valve of canister 12 and provides a conduit to direct the pressurized medicament mist to mouthpiece 20. Delivery chamber 18 further includes upper surface 24 that is used to position the inventive inhaler monitor, as subsequently further described. Mouthpiece 20 includes delivery opening 26 that is in flow communication with delivery chamber 18. Mouthpiece 20 is typically covered by a removable dust cap (not shown).

In use, canister 12 is placed into canister housing 16 of delivery device 14 through opening 22. The user then places his or her mouth over mouthpiece 20 and presses canister 12 downwardly into canister housing 16 to actuate the metering valve of canister 12 and deliver the medicament mist, which the user inhales through opening 26 in mouthpiece 20.

Monitor device 30 is attached over the body of delivery device 14 such that use of inhaler 10 is not compromised.

An electronics unit central device may be attached between the mouthpiece 20 and the lower end of canister 12. The electronics unit central device contains sensors and speakers. The sensors detect various functions, e.g., if a patient is adequately shaking the canister, if a patient is attaching a spacer device to the inhaler, if the medicament is being released by compression of the canister, and if the patient is inhaling the medication too forcefully. The speakers provide oral instructions on proper use, and/or correction of improper use if the sensors detect improper use.

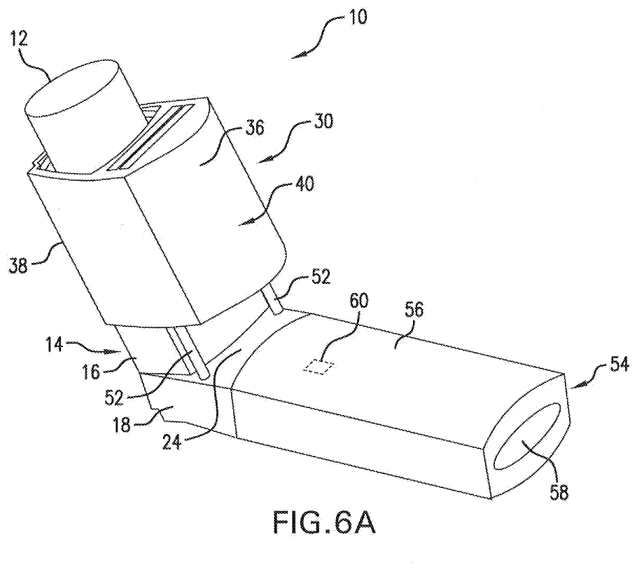

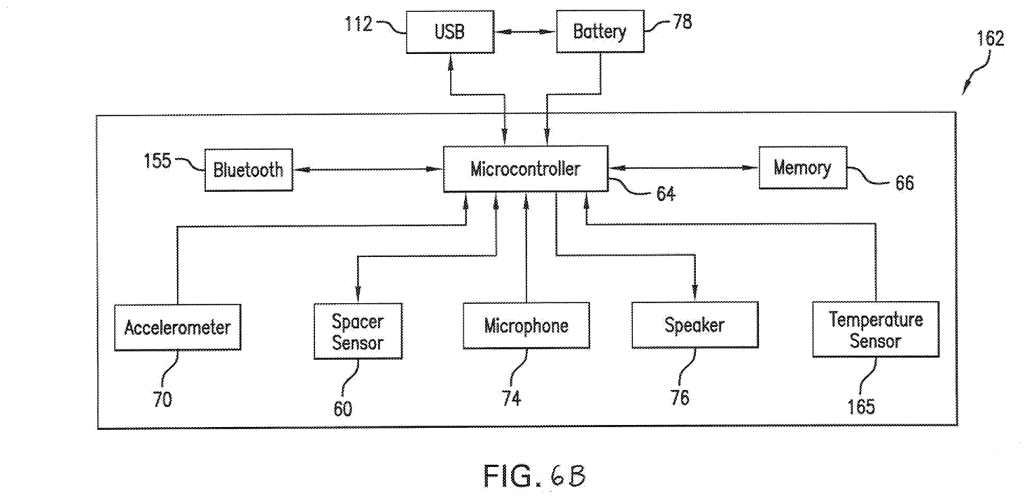

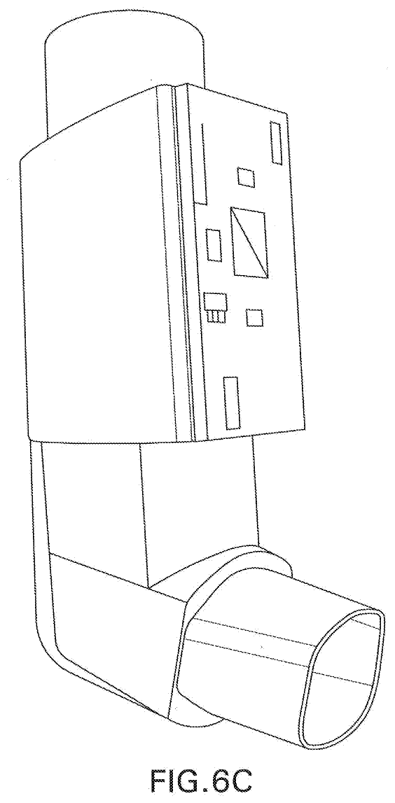

As subsequently described, some clinicians recommend use of a spacer device, termed spacer 54 with inhaler 10 as shown in FIG. 6A. Spacer 54 attaches to mouthpiece 20 and provides a small chamber into which medicament mist from inhaler 10 is delivered before inhalation by the user. Spacers improve delivery of the medicament mist to the lungs. After medicament mist is delivered to spacer 54 medicament mist is briefly suspended within the spacer chamber. Then, the user inhales the medicament mist by placing his or her mouth on mouthpiece 20 of spacer 54 and slowly breathing in. Some spacers include a whistle or other sound emitter that sound if the user inhales too aggressively to facilitate correct user use. Spacers also function to reduce the amount of medicament that settles into the upper airways of the user where it is ineffective, thereby maximizing the amount of medicament that is delivered to the lungs.

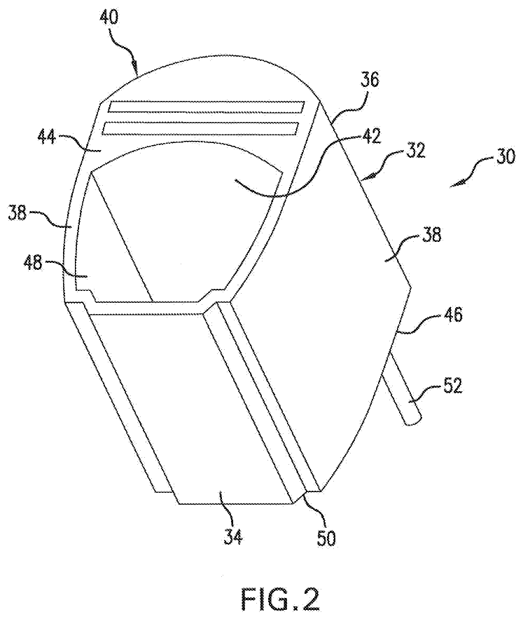

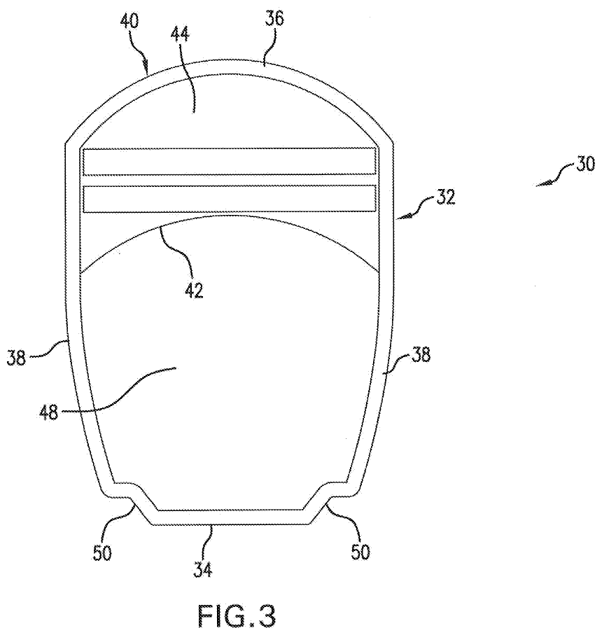

Referring to FIGS. 2, 3, and 4, monitor unit 30 can be attached to and augment the function of a traditional delivery device such as an inhaler. Monitor unit 30 contains embedded electronics that can identify numerous parameters. Monitor unit 30 can identify when a spacer 54 is attached to inhaler 10 as by detection of a magnetic strip, RFID chip or NFC device, collectively 60, embedded into spacer 54. The strip, chip, or device 60 is sufficiently proximate to monitor unit 30 that when monitor unit 30 registers medicament dispersal, monitor unit 30 will either register a use with a spacer 54 or a use without a spacer 54 and transmit notice to a user that the spacer 54 is not attached. Clinicians typically recommend a user shake inhaler 10 before use to ensure medicament is dispersed throughout the propellant to achieve uniform dosing. Monitor unit 30 can identify if the user has properly shaken the device for the appropriate duration before use by using a built-in accelerometer. Monitor unit 30 detects release of the medicament by using sound detection using, e.g., a microphone. Other potential sensors to fulfill this task could include, but are not limited to, a temperature sensor 165, a capacitance sensor, an electronic noise, an ambient light sensor, a pressure sensor, etc. The microphone 74 is set to a threshold determination when a proper inhalation force is exceeded and thus alerts the user. Inhalation with a correct measured force ensures medicament delivery to the lungs. Inhalation that exceeds the correct measured force can result in medicament lodgment at the back of the user's throat. Monitor unit 30 can detect if a user is inhaling the medicament too forcefully because spacer 54 may “whistle” which the microphone on monitor unit 30 will detect as an aberrant sound and thus an incorrect use, with algorithms inherent in these smart sensors. Monitor unit 30 also alerts and interacts with the user to provide reminders of when and how the medication should be taken, and then gives instructions where needed to facilitate appropriate use of both inhaler 10 and spacer 54. Monitor unit 30 also creates compliance reports through a Bluetooth 155 or other communication link with a user's mobile device running a custom application. Monitor unit 30 may have a battery save mode of operation that extends battery life. A single-use battery or a rechargeable battery could be employed.

The inventive system facilitates proper use of delivery device 14 and compliance with the physician prescribed treatment regimen for the particular user. As one example, it provides patient reports to a prescribing physician so remediation and/or counselling in proper use of the delivery device can be initiated, and compliance and efficacy can be evaluated. As another example, it instructs patients on proper self-administration. The training features of the invention are important to help patients understand the proper method for use of the delivery device. Patients and their parents sometimes incorrectly believe that a loud whistle is an indicator of correct inspiratory flow. By including a training module that offers real time patient feedback, there is less room error and enhanced liklihood for proper use of the device and medicine deliviry.

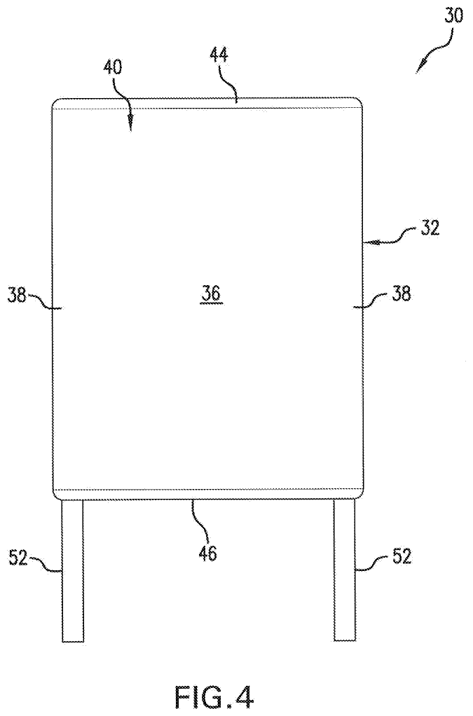

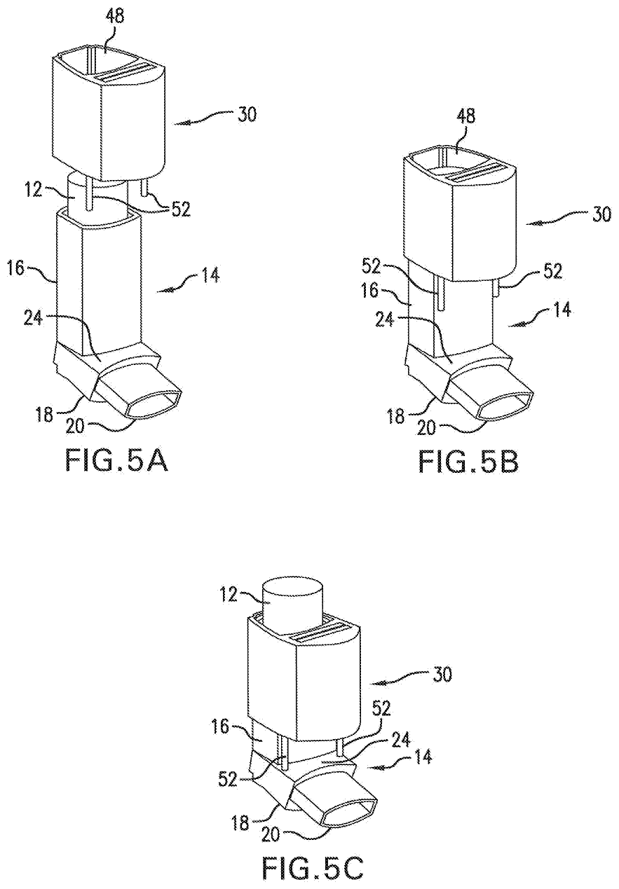

FIGS. 2, 3, and 4 show an inventive monitor unit 30 that generally includes body 32 having rear wall 34, forward wall 36, and side walls 38. Housing 40 is enclosed on three sides by forward wall 36 and side walls 38. Housing 40 includes inner wall 42, upper wall 44, and lower wall 46. Side walls 38, inner wall 42 of housing 40, and rear wall 34 define a central opening 48 configured to receive canister housing 16 shown in FIG. 5 of inhaler 10. Notches 50 are formed along the length of rear wall 34 at its interfaces with side walls 38. Positioning posts 52, one of which is shown in FIG. 2 and the pair shown in FIG. 4, extend from lower wall 46 of housing 40 to properly position monitor unit 30 onto inhaler 10, as subsequently described.

FIGS. 5A-C show how monitor unit 30 is attached to delivery device 14. After canister 12 is inserted into delivery device 14, monitor unit 30 is positioned over canister housing 16 such that central opening 48 of monitor unit 30 receives canister housing 16. Monitor unit 30 is then positioned, e.g., by sliding down, over canister housing 16 until positioning posts 52 engage upper surface 24 of delivery chamber 18. Such positioning ensures that monitor unit electronics can accurately perform their described functions. Notches 50 ensure that monitor unit 30 remains stationary during use.

FIG. 6A shows spacer 54 attached to mouthpiece 20 (shown in FIGS. 5A-C) of delivery device 14. Specifically, spacer 54 includes an outer body 56 that defines a chamber (not shown). The chamber is in flow communication with delivery opening 26 of mouthpiece 20 (FIG. 1) such that medicament mist is delivered from delivery opening 26 to the chamber in outer body 56. Outer body 56 also includes an outlet 58 through which the user inhales the contents present in the internal chamber (not shown) of spacer 54, i.e., the medicament mist from delivery device 14. In embodiments using spacer 54, a magnet, RFID or NFC chip 60 is embedded in an outer wall 56 of spacer 54. The magnet, RFID or NFC chip 60 detects the presence of spacer 54 by monitor unit 30, as subsequently described.

A printed circuit board or integrated circuit device or combination of devices is positioned within housing 40 of monitor unit 30 or delivery device 14 and includes a variety of electrical components. Referring to FIG. 6B, monitor unit 30 includes printed circuit board 62 having a plurality of components mounted thereon, including microcontroller 64, memory 66, transceiver 68, accelerometer 70, Hall-Effect sensor 72, microphone 74, and speaker 76, Bluetooth 155, spacer sensor 60 and temperature sensor 165. Battery 78 and USB port 112 may also be mounted within housing 40 and coupled to printed circuit board 62 to supply power to the components described. Additional active and passive components may be mounted to printed circuit board 62 as part of the electronics needed for the depicted components to function. Some or all of these components may be integrated as an application-specific integrated circuit or other custom device. Several of the described components may be mounted to the structure of housing 40 or monitor unit 30 other than printed circuit board, and Hall-Effect sensor 72 may be replaced with other sensing technology if a device other than magnet is used, i.e., if an RFID chip or an NFC chip is used.

Hall-Effect sensor 72 is mounted near forward wall 36 of monitor unit 30 such that it is positioned adjacent to magnet 60 embedded in outer body 56 of spacer 54 when spacer 54 is attached to delivery device 14. In this manner, Hall-Effect sensor 72 can detect when spacer 54 is attached to delivery device 14. Hall-Effect sensor 72 sends a detection signal to microcontroller 64 upon detecting spacer 54. Microcontroller 64 may respond to receipt of the detection signal by activating certain monitoring functions. For example, microcontroller 64 may activate microphone 74 to detect that medication has been ejected. For example, microcontroller 64 may activate microphone 74 to detect a sound such as a whistle that indicates the user is inhaling too quickly. Microcontroller 64 may also begin recording data assessing the user's correct or incorrect use of device 14. Microcontroller 64 may be active even without spacer 54 being attached to device 14, as users may use device 14 without spacer 54 even in contradiction to a clinician's instructions. FIG. 6C shows views of circuit board 62 with electronic components.

Monitor unit 30 can passively detect the presence of spacer 54 without the need for supplying power to spacer 54, thereby reducing the cost and complexity of spacer 54. While a Hall-Effect sensor is described, in certain applications other sensors may be appropriate. Such sensors include, but are not limited to, contact sensors, optical sensor, infrared (IR) sensors, etc. One of skill will understand that if the sensor is an infared sensor, then multiple configurations of IR transmitter, IR receiver and reflector can be used to transmit and to receive signal.

Accelerometer 70 determines whether the user properly shakes delivery device 14, i.e., canister 12, according to predefined clinician's instructions. Data representing the shaking instructions may be stored in memory 66 and read and interpreted by microcontroller 64. When the user picks up delivery device 14 for use, microcontroller 64 monitors an output signal from accelerometer 70 which indicates when, how vigorously, in what direction, and what duration delivery device 14 is shaken. If delivery device 14 is improperly shaken before use, microcontroller 64 may provide a message to the user by speaker 76. When the user initially picks up delivery device 14, accelerometer 70 may send a wake up signal to microcontroller 64 which transitions microcontroller 64 and other components of monitor 30 from a “sleep” mode to an operational mode. After delivery device 14 is used, microcontroller 64 can place monitor 30 in a sleep mode to preserve power from battery 78 until monitor unit 30 is again awakened by accelerometer 70 or, in alternative embodiments, by pressing an activation button 106. “Sleep” mode may permit battery 78 to supply power to monitor unit 30 for approximately one month before requiring recharge.

Monitor unit 30 can detect when a dose of medicine is delivered by delivery device 14 using microphone 74. Specifically, when delivery device 14 is activated to release a dose of medicine, delivery device 14 makes a distinct noise as pressurized gas is released from canister 12. Microphone 74 receives these sound waves and delivers signals to microcontroller 64, which may be programmed to identify the electronic signals corresponding to a release of pressurized gas from canister 12. In this same manner, microcontroller 64 can detect when canister 12 malfunctions, e.g., is clogged, by detecting an abnormal series of electronic signals from microphone 74 when canister 12 is activated.

Speaker 76 can be used to audibly instruct a user through the steps of properly using their delivery device, or in correcting their technique if one or more sensors determines that a step was not correctly performed.

Spacer 54 may include a whistle that sounds when the user inhales medication from spacer 54 too forcefully. Microphone 74 can also detect this noise, provide corresponding signals to microcontroller 64, and thereby cause microcontroller 64 to provide additional feedback to the user by speaker 76, i.e., an audible message to inhale less forcefully, and/or create a record of the event in memory 66 for later review by a clinician during an in-person visit, or send a message to a clinician by transceiver 68 in real time.

Around mouthpiece 20, 22 there is capacitive sensor 39. Capacitive sensor 39 detects when the patient's mouth is on mouthpiece 20, 22 to determine whether a complete seal is formed around mouthpiece 20, 22. Capacitive sensor 39 communicates whether a seal is created, and if it is not, then capacative sensor 39 communicates a message to the patient about how to remedy any problems.

Mouthpiece 20, 22 may also include temperature sensor 65 to determine if a dose of medication was given. Temperature sensor 65 detects a reduction in temperature when the patient inhales medicament mist due to cooler air being drawn across into the delivery device during inhalation. The inventive array of sensors may also be configured to measure the time between actuation and the temperature reduction to determine if the proper method of inhalation was used.

Monitor 30 may be paired with a mobile device such as a smart phone, tablet, etc. The mobile device may run one or more applications. One application may be configured for clinicians who can program the settings onto the user's mobile device, such as prescribed medication name, the number of puffs and doses per day, the dosing schedule, etc. The application may allow the user, and/or the user's caregivers, to set alarms to remind the user of the dosage schedule, and to set monitor 30 to provide regular prompts/instructions by speaker 76 or to only record usage data for upload to a clinician.

A mobile phone/tablet application may be used to remotely program the electronics unit central device, and to record and to transmit data to a repository which can be accessed by a clinician or researcher. The clinician will then know if the patient is receiving the prescribed doses on the prescribed schedule and if each dose is being administered appropriately. In follow-up, a communication chat-box can automatically send a message to the patient alerting the patient to the problem, e.g., “Your last administered dose was not delivered optimally.” The patient is also able to track his/her uses, determine how closely he/she is following clinician instructions, and to initiate reminders to help follow and maintain a specific dosage schedule.

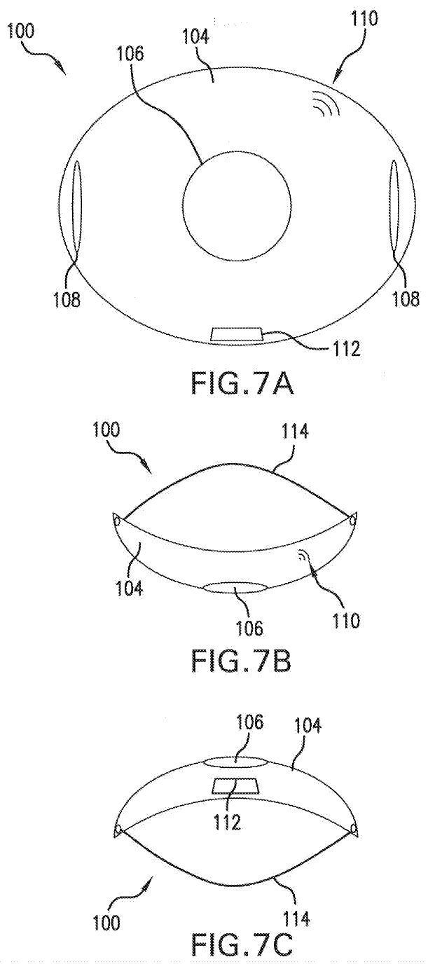

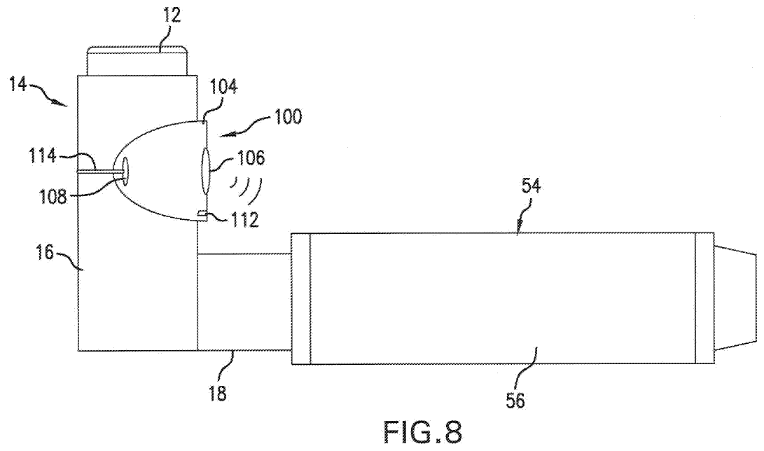

FIGS. 7A-C show monitor 100 configured to attach to a delivery device 14 as previously described. Monitor 100 includes body 104, central button 106 disposed in a central region of body 104, slits 108 disposed adjacent opposed edge regions of body 104, speaker 110 mounted to body 104, and USB port 112 mounted to body 104. Elastic band 114 extends between slits 108 and is used to attach monitor 100 to canister housing 16 of delivery device 14, shown in FIG. 8.

All components, i.e., sensors, processor, and speaker, may be embedded within the delivery device itself, rather than in a removable monitor unit or spacer, obviating the need for an additional attachment, providing less bulk for a patient to both carry with them and to remember to attach to their device.

FIG. 8 shows delivery device 14 with attached spacer 54. Delivery device 14 includes canister 12, canister housing 16, and delivery chamber 18. Spacer 54 is attached to mouthpiece (not shown) of delivery device 14. Monitor 100 is attached to delivery device 14 by stretching elastic band 114 and placing body 104 and elastic band 114 onto canister housing 16. Elastic band 102 is similarly positioned onto spacer 54 at an end of adjacent delivery chamber 18. In this manner, a sensor (not shown) on body 104 can detect the presence of RFID/NFC chip 116 and therefor the presence of spacer 54. Spacer 54 includes an IR sensor to signal to the device. The use of IR allows it to communicate to devices that are not within NFC or RFID range. This sensor can either be an IR transmitter or an IR transmitter/receiver. If the main device has the IR transmitter or the transmitter/receiver, then the spacer can include the receiver or reflector, respectively. This spacer alaso includes a battery to provide power to the sensors. An accelorometer in the spacer allows it to recognize movement and “wake” the rest of the sensors, permitting the spacer to “sleep”, thus conserving battery power.

One embodiment does not include a battery in the spacer, but instead contains wires that connect with contacts near the mouthpiece of the device to both get power and to signal that a spacer is present.

Monitor 100 may further include a rechargeable battery (not shown) and any or all of the other components of monitor 30 previously described.

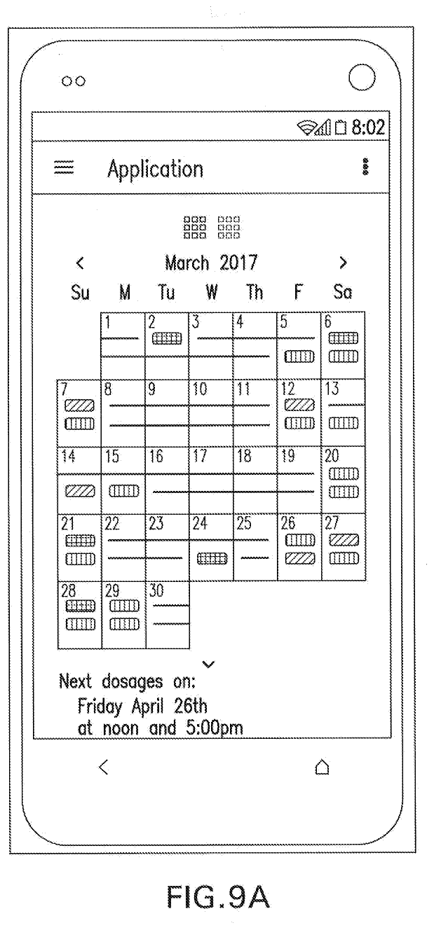

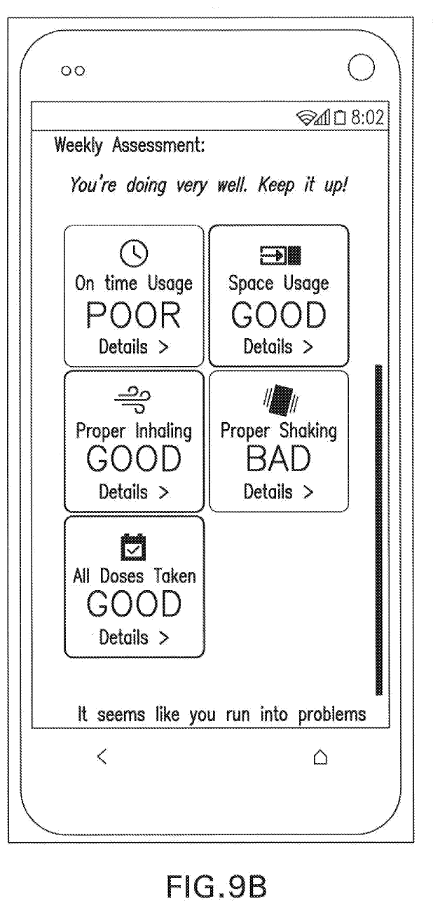

FIGS. 9A-B are sample screen displays that show user compliance, and feedback on medication use. For simplicity and quick review, color codes may indicate levels of user compliance and treatment efficacy. FIGS. 9A-B are exemplary but not limiting illustrations of the mobile application for patient use. FIG. 9A shows a month calendar view that allows a user to track his or her daily usage, usage trends such as a problem with compliance on weekends or holidays, etc. FIG. 9B shows the quality of the uses over the previous weeks, helping users to learn how to improve their use of the inhaler. By highlighting common misuse problems, e.g., improper inhaler shaking before use, the user receives feedback on ways to improve and optimize their treatment. The application also provides details as to why use was improper, e.g., it will report a continual practice of inhalation exceeding a desired force, and it will suggest one or more ways to remediate the improper use.

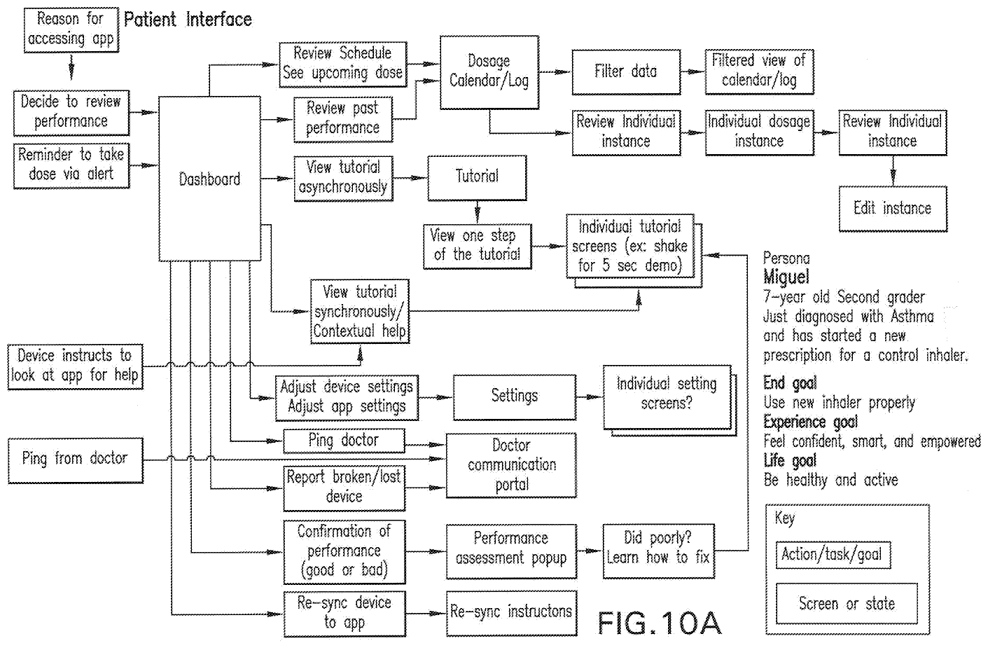

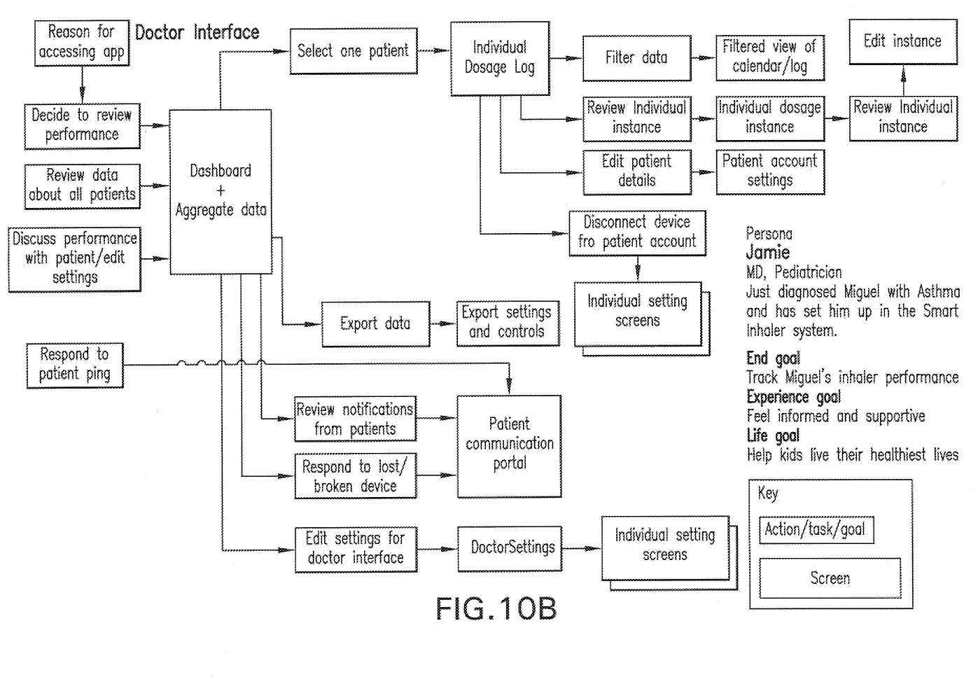

FIG. 10A shows one exemplary algorithm for the user interface of a phone application. The upper path shows how a user can check the application for a calendar and individual usage statistics. The bottom path shows what happens during use of the inhaler. The lower path shows how the application recognizes issues during use, and provides user feedback and/or a tutorial for improved use.

FIG. 10B is a chart representation of one exemplary algorithm for a clinician interface of the application. The upper path indicates a clinician's access to a user's information and the method by which the clinician can move into and view that information. The lower path shows how the clinician can enter user-specific instructions.

FIGS. 10A and 10B are exemplary but not limiting illustrations of the capabilities and software algorithms for a user's mobile device application.

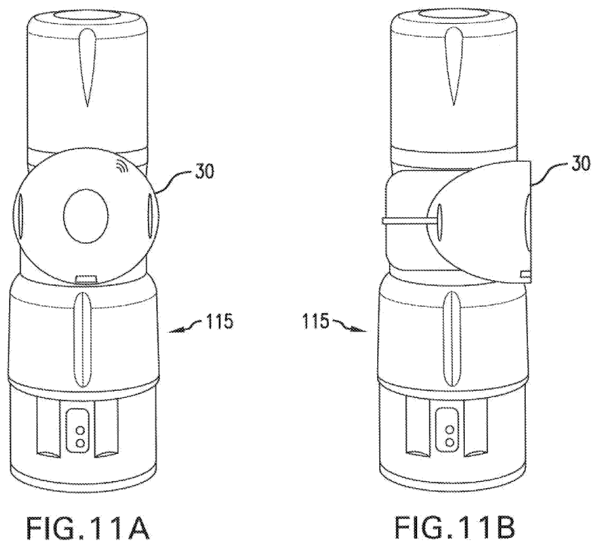

FIGS. 11 and 12 demonstrate that the device can be used in more than just metered dose inhalers. FIG. 11A and FIG. 11B illustrate monitor unit 30 attached to a dry particle inhaler 115. FIG. 12 illustrates monitor unit 30 attached to a nebulizer 120.

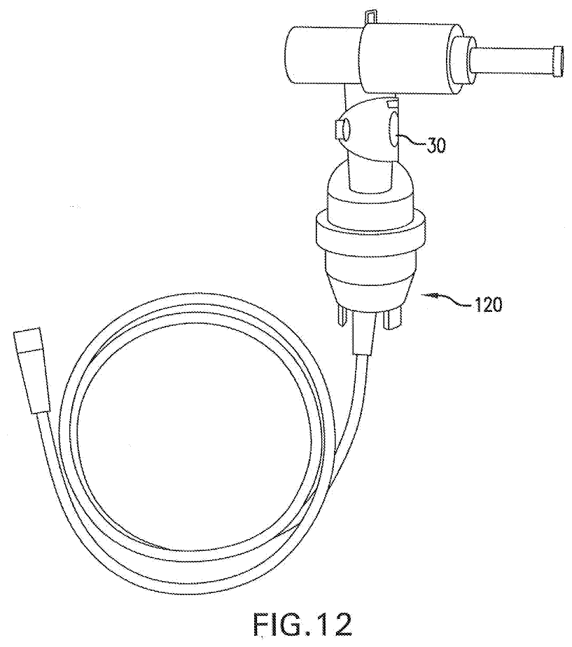

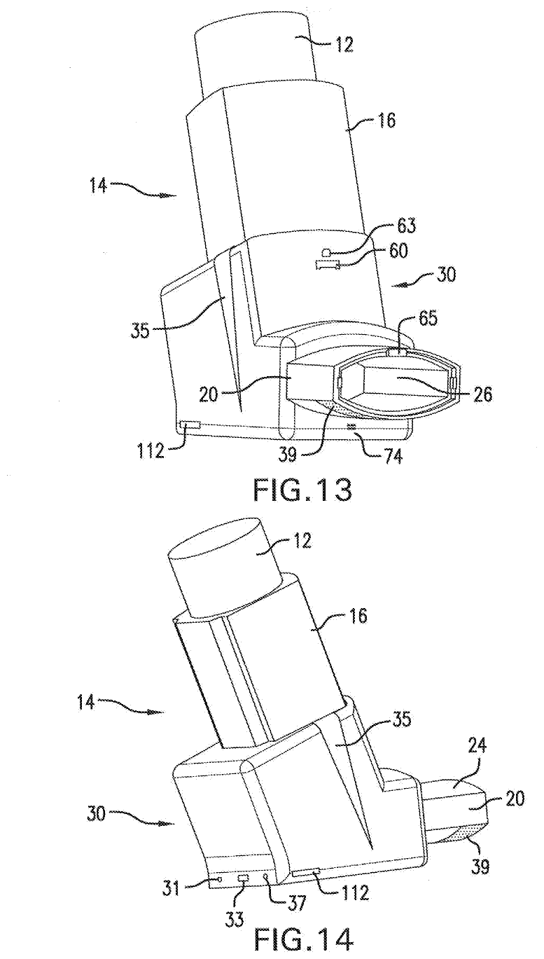

FIG. 13 and FIG. 14 shows perspective views of a preferred embodiment of the invention. Delivery device 14 generally includes a canister housing. Canister 12 is placed in canister housing 16. When canister 12 is actuated, a medicament aerosol is released from canister 12 in a controlled manner to deliver a specific quantity of the medicament to the user through mouthpiece 20. Delivery device 14 includes a capacitive sensor 39 on the lower surface of mouthpiece 20 opposite upper surface 24. Capacitive sensor 39 detects if a mouth of a user has formed a sufficient seal around mouthpiece 20. Monitor device 30 is attached over the body of delivery device 14. Monitor device 30 includes material 35 capable of stretching to widen the monitor unit central opening to facilitate attachment of the monitor unit to the medication delivery device through the monitor unit central opening. Monitor device 30 further includes light sources 31, 37. One or more light sources such as light emitting diodes may be used to indicate charge level and/or to provide user feedback. Monitor device 30 also includes USB port 33 and memory card insertion port 112.

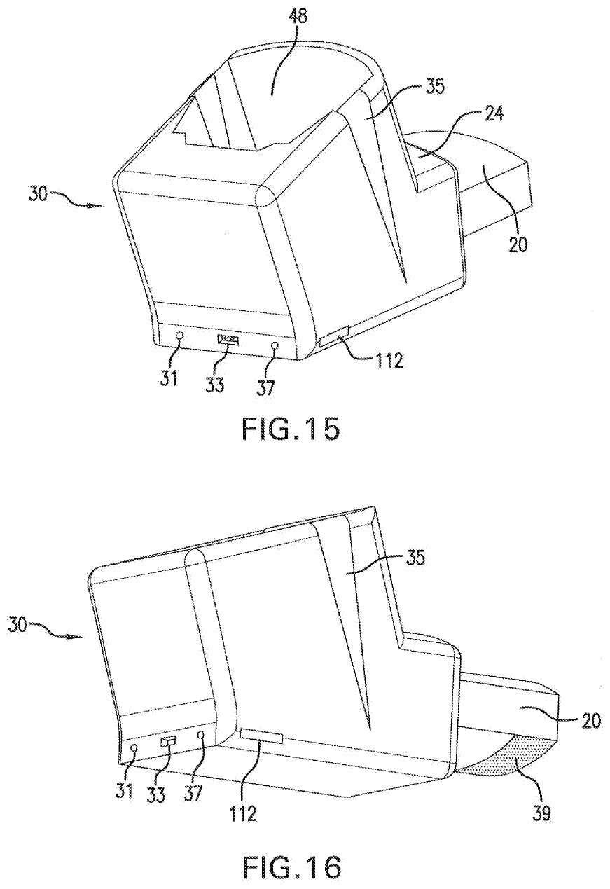

FIG. 15 and FIG. 16 show perspective views of an embodiment of the invention. Monitor device 30 includes central opening 48 configured to receive delivery device 14. Monitor device 30 includes a material 35 capable of stretching to widen the monitor unit central opening to facilitate attachment of the monitor unit to the medication delivery device through the monitor unit central opening. Monitor device 30 further includes light sources 31, 37, USB port 33 and memory card insertion port 112.

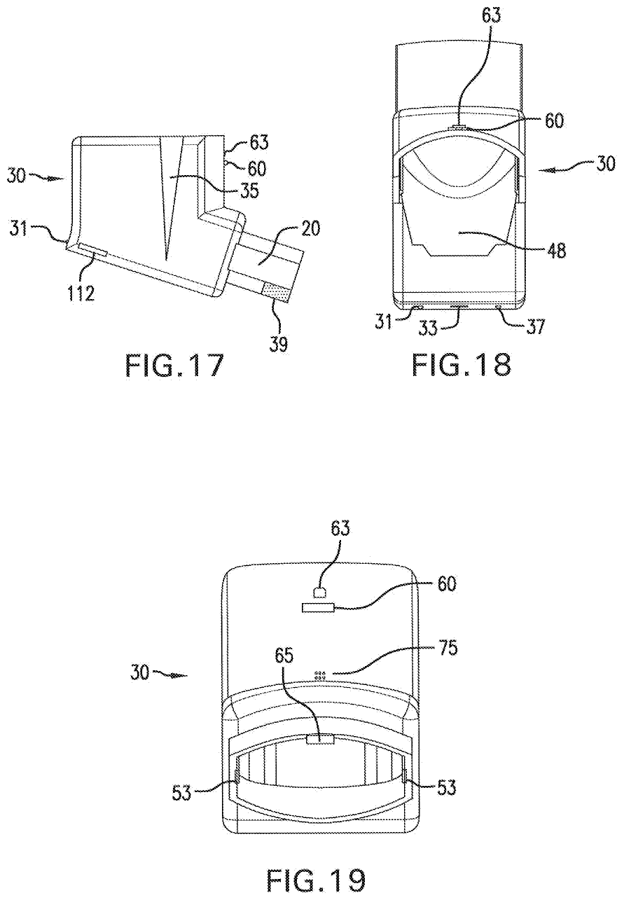

FIG. 17 shows an orthogonal right side view of an embodiment of the invention. Monitor device 30 includes a material 35 capable of stretching to widen the monitor unit central opening to facilitate attachment of the monitor unit to the medication delivery device through the monitor unit central opening. Monitor device 30 also includes reflector 63 and magnet, RFID or NFC chip 60. Monitor device 30 further includes light sources 31 and memory card insertion port 112. Capacitive sensor 39 is shown on mouthpiece 20.

FIG. 18 shows an orthogonal top side view of an embodiment of the invention. Monitor device 30 includes a central opening 48 configured to receive delivery device 14. Monitor device 30 also includes reflector 63 and magnet, RFID or NFC chip 60. Monitor device 30 further includes light sources 31, 37 and USB port 33.

FIG. 19 shows an orthogonal front side view of an embodiment of the invention. Monitor device 30 includes reflector 63 and magnet, RFID or NFC chip 60. Monitor device 30 further includes speaker 75, temperature sensor 65 and contacts points 53.

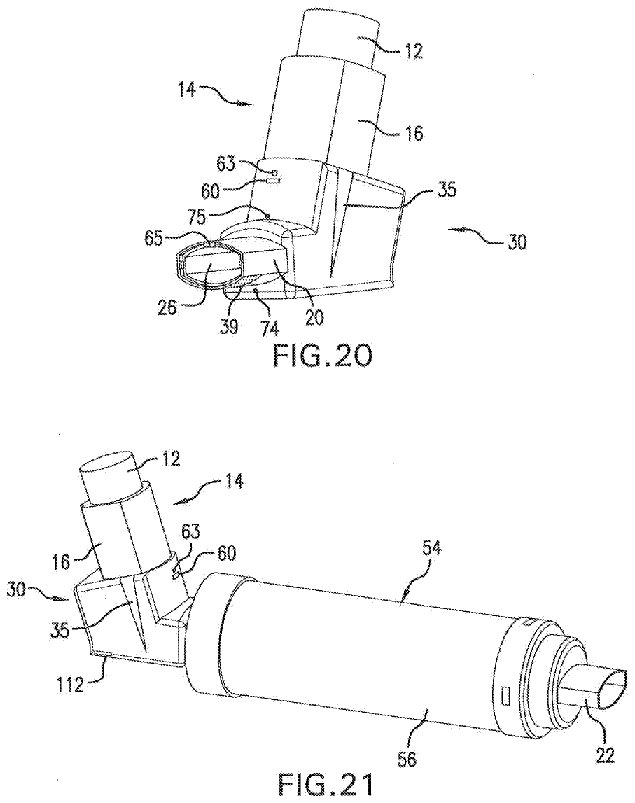

FIG. 20 shows a perspective view of an embodiment of the invention. Monitor device 30 is engaged to delivery device 14. Monitor device 30 includes reflector 63 and magnet, RFID or NFC chip 60. Monitor device 30 further includes speaker 75, temperature sensor 65 and microphone 74. Capacitive sensor 39 is shown on mouthpiece 20.

FIG. 21 shows a perspective view of an embodiment of the invention. Monitor device 30 is engaged to delivery device 14 and to spacer 54. Canister 12 is placed in canister housing 16. When canister 12 is actuated, a medicament aerosol is released from canister 12 in a controlled manner to deliver a specific quantity of the medicament to the user. Monitor device 30 includes a material 35, reflector 63 and magnet, RFID or NFC chip 60. Monitor device 30 further includes memory card insertion port 112.

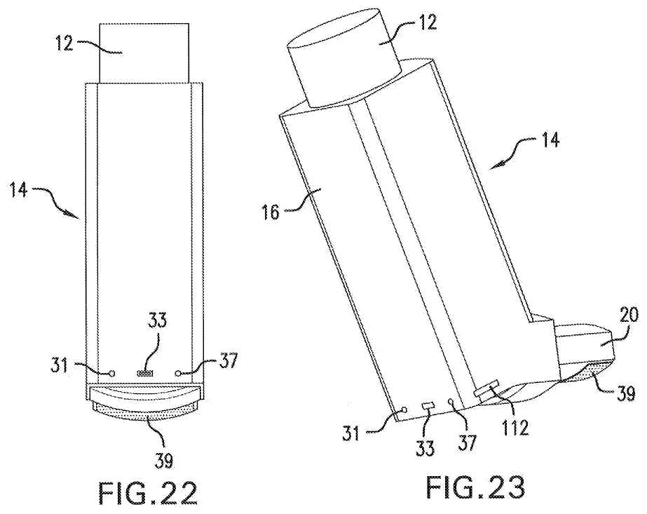

FIG. 22 shows an orthogonal rear side view of an embodiment of the invention and FIG. 23 shows a perspective view of an embodiment of the invention. Canister 12 is placed in canister housing 16 of delivery device 14. Delivery device 14 further includes light sources 31, 37, a USB port 33 and a memory card insertion port 112. Capacitive sensor 39 is shown on mouthpiece 20.

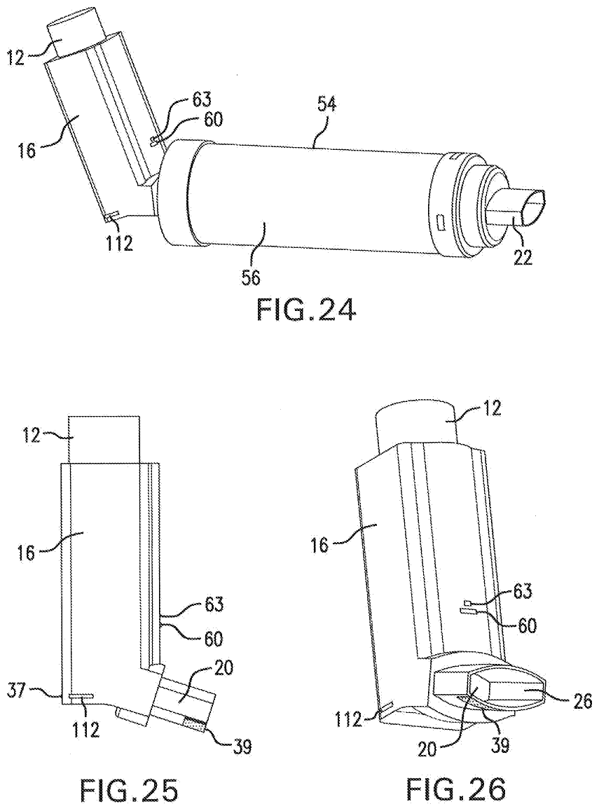

FIG. 24 shows a perspective view of an embodiment of the invention in which all sensing, data storage, data processing, and communication components of the monitor unit have instead been integrated into the canister housing body, FIG. 25 shows an orthogonal right side view of an embodiment of the invention, and FIG. 26 shows a perspective view of an embodiment of the invention. Canister 12 is placed in canister housing 16 of delivery device 14. Delivery device 14 further includes light source 37 and a memory card insertion port 112. Capacitive sensor 39 is shown on mouthpiece 20. Delivery device 14 includes reflector 63 and magnet, RFID or NFC chip 60. As shown in FIG. 24, spacer 54 is coupled to delivery device 14.

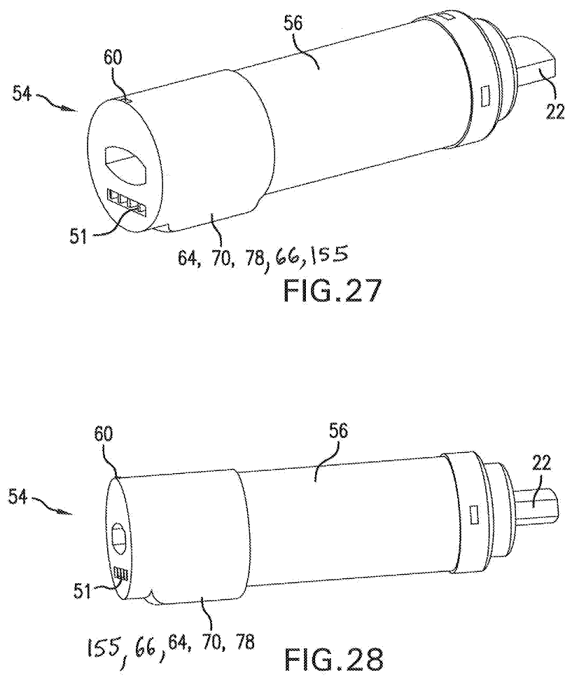

FIG. 27 and FIG. 28 show perspective views of the same embodiment of the invention. Spacer 54 includes whistle 51, magnet, RFID or NFC chip 60, reflector 63 and IR receiver-emitter or RFID chip or magnet 64, accelerometer 70, battery 78, Bluetooth 155, data storage/memory 66, and microcontroller 64.

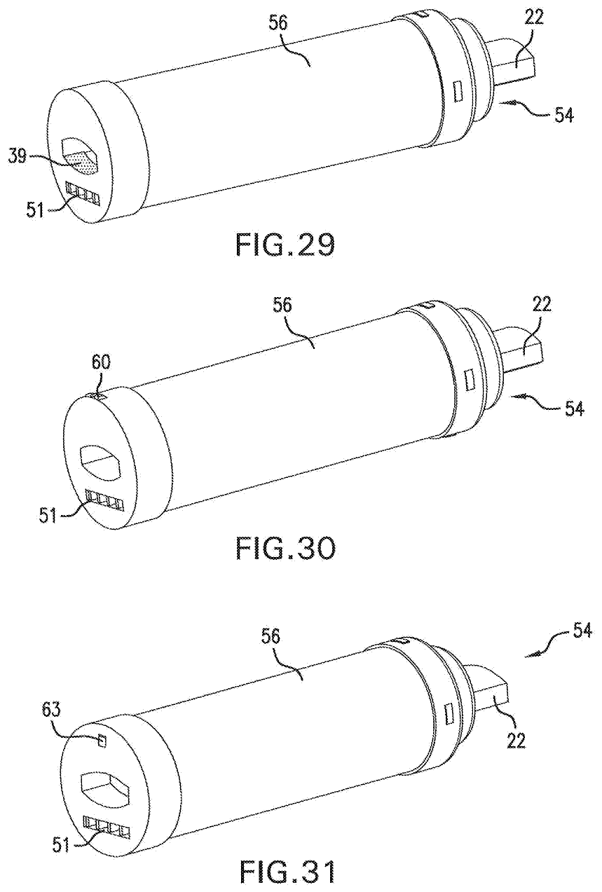

FIG. 29, FIG. 30 and FIG. 31 each show perspective views of alternate and distinctembodiments of the invention. Spacer 54 includes whistle 51, capacitive sensor/material 39, magnet, RFID or NFC chip 60 and reflector 63.

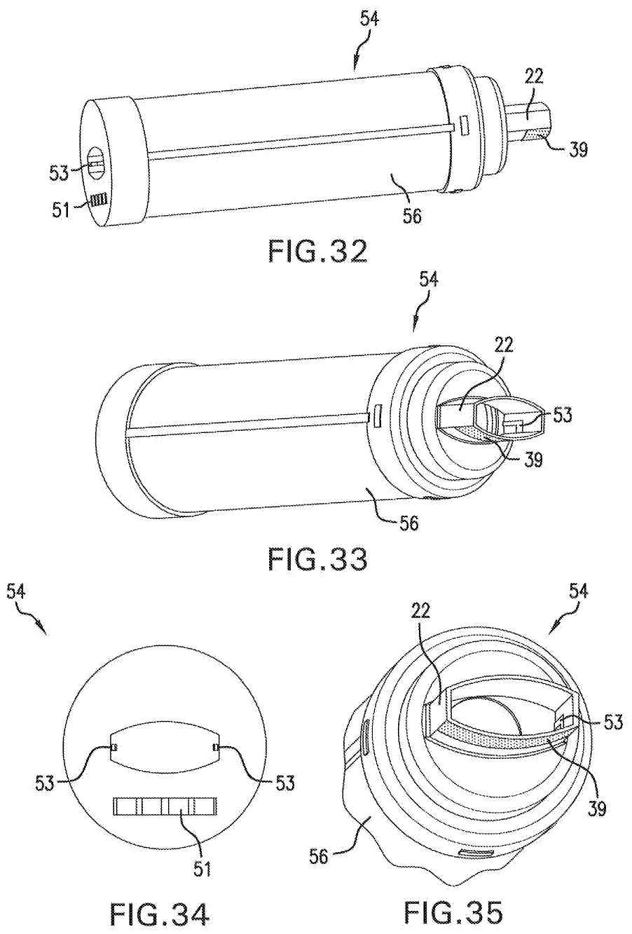

FIG. 32, FIG. 33, and FIG. 35 show perspective views of another alternate and distinct embodiment of the invention. FIG. 34 shows an orthogonal rear side view of the embodiment of the invention depicted in FIG. 32, FIG. 33 and FIG. 35. Spacer 54 includes whistle 51, contact point/contact wires 53 and capacitive sensor 39. Capacitive sensor/material (such as but not limited to graphite) 39 in opening of spacer 54 may interact with capacitive sensor /material 39 on the mouthpiece of delivery device to determine whether spacer 54 is present.

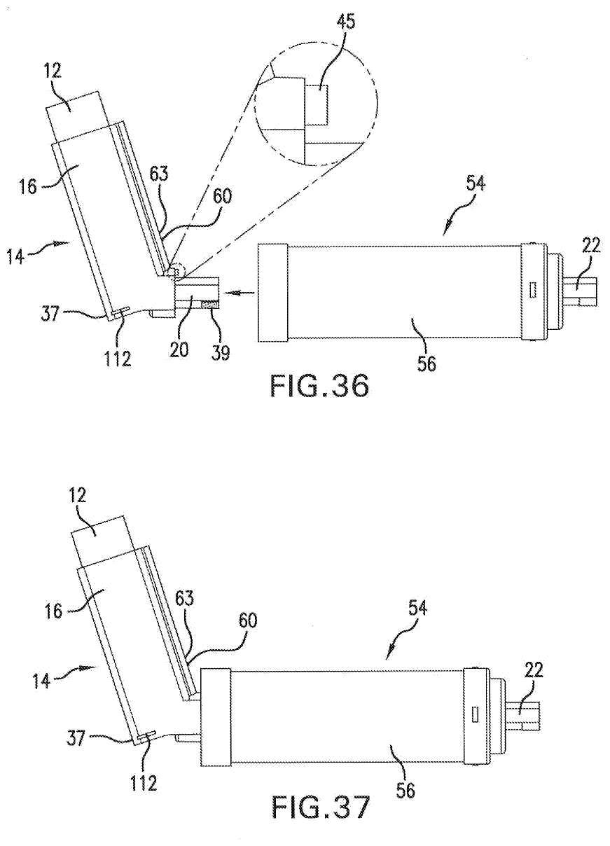

FIG. 36 shows an exploded view of an embodiment of the invention. Canister 12 is placed in canister housing 16 of delivery device 14. Delivery device 14 further includes light source 37, a memory card insertion port 112. Capacitive sensor 39 is shown on mouthpiece 20. Delivery device 14 includes reflector 63 and magnet, RFID or NFC chip 60. The exploded view shows mechanical button 45 on delivery device 14. Spacer 54 couples to delivery device 14 depressing mechanical button 45 which can be sensed by the inventive system. FIG. 37 shows an orthogonal right side view of an embodiment of the invention with delivery device 14 coupled to spacer 54 and mechanical button 45 (not shown) depressed.

It should be understood that in any of the described embodiments, various features may readily be incorporated. For example, the monitors may be configured for wireless charging, be water resistant, include a display, etc.

The connecting lines shown in the figures represent exemplary functional relationships and/or physical couplings between the elements. One skilled in the art will appreciate that many alternative and/or additional functional relationships or physical connections may be present in a system in use, with benefits, advantages, solutions to problems, and any elements that may cause same are not to be construed as critical, required, or essential.

Various modifications and additions can be made to the embodiments disclosed herein without departing from the scope of the disclosure. For example, while the embodiments described above refer to particular features, the scope of this disclosure also includes embodiments having different combinations of features and embodiments that do not include all of the described features. Thus, the scope of the present disclosure is intended to embrace all such alternatives, modifications, and variations as fall within the scope of the claims, together with all equivalents.

What is claimed is:

1. A medication delivery system that combines user input and sensor data to provide the user with auditory training, resulting in facilitating the user's mastery of a technique to administer a medication.

2. The system of claim 1 where the medication is administered by a route selected from the group consisting of inhalation, oral, intranasal, transdermal, subcutaneous injection, intramuscular injection, intravenous infusion, enema, rectal, bladder catheter, pump, intrathecal, and combinations thereof.

3. A medication delivery system comprising a medication delivery device, and a monitor attached to the medication delivery device, the monitor including a speaker configured to provide, in real-time, at least one of medication use prompts, reminders, and feedback to at least the user and optionally also to a clinician caring for the user.

4. The medication delivery system of claim 3 further comprising at least a first processor that is programmed in real time by the clinician with patient specific instructions or altered patient specific instructions.

5. The medication delivery system of claim 4 where the instructions are selected from the group consisting of medication type, dose quantity, dose volume, dose time, dose frequency, and combinations thereof.

6. The medication delivery system of claim 5 further comprising a second processor that compares new instructions with previous instructions and transmits the comparison to a mobile device application.

7. The medication delivery system of claim 3 where the medication delivery device is an inhaler comprising a canister housing and a user inhaler device,
a spacer coupled to the user inhaler device, the spacer including at least one of a magnet, a radiofrequency identification (RFID) chip, or a nearfield communication (NFC) device; and
where the monitor is removeably attached to the canister housing of the medication delivery device, the monitor including a sensor configured to sense the presence of the magnet, RFID chip, or NFC device of the spacer and to indicate the presence of the sensor to a microcontroller mounted to the monitor.

8. The medication delivery system of claim 7 where the inhaler is a metered dose inhaler.

9. The medication delivery system of claim 7 where the inhaler is a non-metered dose inhaler.

10. A medication delivery system comprising a medication delivery device, and a monitor attached to the medication delivery device, the monitor including a motion sensor configured to detect at least one motion characteristic of use of the medication delivery device.

11. A medication delivery system comprising a medication delivery device, and a monitor attached to the medication delivery device, the monitor including a motion sensor configured to detect use of the medication delivery device and respond to a signal indicating use of the medication delivery device by transitioning from a sleep mode to an operational mode.

12. A medication delivery system comprising a medication delivery device, and a monitor attached to the medication delivery device, the monitor including a microphone configured to detect use of the medication delivery device.

13. A medication monitoring system comprising
a medication inhaler;
a spacer coupled to the medication inhaler, the spacer comprising at least one magnet, radiofrequency identification (RFID) chip, a nearfield communication (NFC) device, or means to engage the spacer;
a plurality of sensors configured to detect the spacer when the inhaler is in use;
at least one accelerometer;
at least one microphone;
at least one sensor;
at least one memory unit;
at least one speaker;
at least one transmitter; and
at least one processor configured to receive data from the plurality of sensors.

14. The medication monitoring system of claim 13 where the sensor is selected from the group consisting of a capacitance sensor, a thermometer, an infrared light detector, an ambient light sensor, a pressure sensor, a humidity sensor, an impedance sensor, and combinations thereof.

15. The medication monitoring system of claim 13 where at least one of the plurality of sensors senses when the medication is delivered from the inhaler.

16. The medication monitoring system of claim 13 where at least one of the plurality of sensors is an accelerometer that detects qualitative and quantitative inhaler shaking prior to medication delivery from the inhaler.

17. The medication monitoring system of claim 13 where at least one of the plurality of sensors detects the corresponding magnet or RFID chip or NFC device located in the spacer.

18. The medication monitoring system of claim 13 where the at least one processor is programmed in real time with patient specific instructions or altered patient specific instructions.

19. The medication monitoring system of claim 18 where the instructions are selected from the group consisting of type of medication, dose quantity, dose volume, inhaler shaking, spacer presence, time of dose, and combinations thereof, and the processor generates a comparison of the instructions with data from the plurality of sensors and transmits the comparison to a mobile device application.

20. The medication monitoring system of claim 13 where the transmitter is configured to receive data from the first processor and transmit the received data to a phone application and to the clinician.

21. A method to determine correct use of an inhaler by a user, the method comprising
providing a user with an inhaler comprising a medication monitoring device, the device comprising a plurality of sensors configured to transmit data to at least one processor,
programming the at least one processor with user-specific instructions;
determining whether the data concurs with the user-specific instructions,
storing the data and alerting the user and/or clinician with nonconcurrance; and
transmitting the data comparison, using a transmitter, to the user and clinician, where the data comparison indicates instruction compliance or non-compliance and provides suggestions for achieving compliance or affirmations of compliance.

22. The method of claim 21 where the user-specific instruction are from at least one of a clinician or a manufacturer.

23. The method of claim 21 where the clinician instructions are stored in at least one memory.

24. The method of claim 21 further including a first processor retrieving the stored data about medication dose administration time from the at least one memory.

25. The method of claim 24 where the processor transmits the data to the at least one transmitter to transmit a reminder to the user's mobile device.

26. The method of claim 21 where the plurality of sensors transmit recorded data to the at least one processor, and the processor stores the transmitted data in at least one memory.

27. The method of claim 21 where the at least one transmitter transmits the recorded data to both the user's mobile device and to an on-line data repository for clinician and/or researcher access.

28. The method of claim 21 during data gathering and recordation by the sensors, the at least one processor compares the recorded data with the programmed instructions and determines if the recorded data aligns with the programmed instructions.

29. The method of claim 21 where non-aligned data results in the at least on processor transmitting feedback to the user through the at least one speaker.

30. A monitor unit configured to be coupled to a medication delivery device, the monitor unit comprising:
a monitor unit body and a monitor unit central opening;
a material capable of stretching to widen the monitor unit central opening to facilitate attachment of the monitor unit to the medication delivery device through the monitor unit central opening;
at least one light source that performs a function selected from the group consisting of power indication or visual feedback to a user;
a USB port to receive a USB cable;
a port to receive a memory card;
a sensor to detect if a mouth of a user has formed a seal around a mouthpiece;
a reflector to facilitate communication between an infrared transmitter and an infrared receiver;
at least one of a magnet, an infrared transmitter-emitter, a radiofrequency identification (RFID) chip or a mechanical button to detect a presence of a spacer;
a speaker to provide auditory feedback to a user; and,
at least one microphone to determine if a user is inhaling with proper force and/or to detect dose release.

31. The monitor unit of claim 30, where the monitor unit further comprises at least one contact for supplying battery power to a spacer.

32. A medication delivery device comprising:
at least one light source that performs a function selected from the group consisting of power indication or visual feedback to a user;
a USB port to receive a USB cable;
a port to receive a memory card;
a sensor to detect if a mouth of a user has formed a seal around a mouthpiece;
a reflector to facilitate communication between an infrared transmitter and an infrared receiver;
at least one of a magnet, an infrared transmitter-emitter, a radiofrequency identification (RFID) chip a mechanical button or a capacitive sensor to detect a presence of a spacer;
a speaker to provide auditory feedback to a user; and,
at least one microphone to determine if a user is inhaling with proper force and/or to detect dose release.

33. The medication delivery device of claim 32, further comprising a temperature sensor.

34. A spacer configured to be coupled to a medication inhaler, the spacer comprising:
at least one magnet, radiofrequency identification (RFID) chip, a nearfield communication (NFC) device or a capacitive material.

35. The spacer of claim 34, further comprising an infrared transmitter/emitter.

36. The spacer of claim 34, further comprising a battery.

37. The spacer of claim 34, further comprising an accelerometer.

38. The spacer of claim 34, further comprising a capacitive sensor.

39. The spacer of claim 34, further comprising an infrared reflector.

40. The spacer of claim 34, further comprising a microcontroller.

41. The spacer of claim 34, further comprising Bluetooth.

42. The spacer of claim 34, further comprising data storage.

43. A method to determine correct use of an inhaler by a user, the method comprising
providing a user with an inhaler, the inhaler comprising a plurality of sensors configured to transmit data to at least one processor,
programming the at least one processor with user-specific instructions;
determining whether the data concurs with the user-specific instructions,
storing the data and alerting the user and/or clinician with nonconcurrance; and
transmitting the data comparison, using a transmitter, to the user and clinician, where the data comparison indicates instruction compliance or non-compliance and provides suggestions for achieving compliance or affirmations of compliance.

44. A method to determine proper dose received and proper technique by a user, the method comprising
providing a user with a spacer to use with the inhaler, the spacer comprising a plurality of sensors configured to transmit data to at least one processor,
programming the at least one processor with user-specific instructions;
determining whether the data concurs with the user-specific instructions,
storing the data and alerting the user and/or clinician with nonconcurrance; and
transmitting the data comparison, using a transmitter, to the user and clinician, where the data comparison indicates instruction compliance or non-compliance and provides suggestions for achieving compliance or affirmations of compliance.
